(19) United States
(12) Patent Application Publication
(10) Pub. No.: US 2024/0000717 A1
(43) Pub. Date: Jan. 4, 2024

(54) DRY POWDER ANTIVIRAL COMPOSITIONS AND THEIR USE FOR TREATING VIRAL INFECTION

(71) Applicant: Nano PharmaSolutions, Inc., San Diego, CA (US)

(72) Inventors: Kay Olmstead, Escondido, CA (US); Saeyeon Lee, Seongnam (KR)

(21) Appl. No.: 18/253,455

(22) PCT Filed: Nov. 18, 2021
(86) PCT No.: PCT/US2021/059799

§ 371 Date: May 18, 2023

Related U.S. Application Data
(60) Provisional application No. 63/116,108, filed on Nov. 19, 2020.

(51) Int. Cl. A61K 9/50; A61K 31/53; A61K 9/48; A61K 31/688

(52) U.S. Cl. CPC A61K 9/5015; A61K 31/53; A61K 9/4858; A61K 31/688

(57) ABSTRACT

Provided herein are dry powder antiviral compositions, each comprising a microparticle of a pharmaceutically acceptable excipient and nanoparticles of an antiviral, wherein the surface of the microparticle is coated with the nanoparticles. Also provided herein are methods of their use for treating, preventing, or ameliorating one or more symptoms of a viral infection.

### CROSS REFERENCE TO RELATED APPLICATION

This application claims the benefit of the priority of U.S. Provisional Application No. 63/116,108, filed Nov. 19, 2020; the disclosure of which is incorporated herein by reference in its entirety.

### FIELD

Provided herein are dry powder antiviral compositions, each comprising a microparticle of a pharmaceutically acceptable excipient and nanoparticles of an antiviral, wherein the surface of the microparticle is coated with the nanoparticles. Also provided herein are methods of their use for treating, preventing, or ameliorating one or more symptoms of a viral infection.

### BACKGROUND

Coronavirus disease 2019 (COVID-19) is caused by severe acute respiratory syndrome coronavirus-2 (SARS-CoV-2). Gorbalenya et al., Nat. Microbiol. 2020, 5, 536-44; Zhang et al., Science 2020, 368, 409-12. On Mar. 11, 2020, the World Health Organization declared COVID-19 a global pandemic. Zhang et al., Science 2020, 368, 409-12; Dai et al., Science 2020, 368, 1331-5. By mid-July 2020, COVID-19 has spread almost to every corner of the world. By mid-November 2020, there are over 55 million confirmed cases and over 1.3 million confirmed deaths globally. The COVID-19 pandemic poses as a great public health threat to the world.

COVID-19 can be fatal, especially to older people and those with pre-existing medical conditions. Singhal, Indian J. Pediatr. 2020, 87, 281-6; Shahid et al., J. Am. Geriatr. Soc. 2020, 68, 926-9. Common symptoms of COVID-19 include fever or chills, cough, shortness of breath or difficulty breathing, fatigue, muscle or body aches, headache, loss of taste or smell, sore throat, congestion or runny nose, nausea or vomiting, and diarrhea. Id. Currently, remdesivir is the only FDA-approved drug for COVID-19. Clinically, remdesivir is administered intravenously only. Remdesivir is not suitable for oral delivery because of its poor hepatic stability. Very recently, the largest clinal trial on remdesivir's efficacy run by the World Health Organization showed that the drug had little or no effect on 28-day mortality in hospitalized COVID-19 patients. Pan et al., “Repurposed antiviral drugs for COVID-19—interim WHO SOLIDARITY trial results,” MedRxiv 2020. Therefore, there is an urgent need for an effective therapy to combat the COVID-19 pandemic.

### SUMMARY OF THE DISCLOSURE

Provided herein is a coated particle comprising: (i) a microparticle that comprises a pharmaceutically acceptable excipient, and (ii) nanoparticles of an antiviral; wherein the surface of the microparticle is coated with the nanoparticles.

Also provided herein is a pharmaceutical composition, in one embodiment, a dry powder antiviral composition, comprising coated particles, each particle comprising: (i) a microparticle that comprises a pharmaceutically acceptable excipient, and (ii) nanoparticles of an antiviral; wherein the surface of the microparticle is coated with the nanoparticles.

Additionally, provided herein is a method of preparing coated particles, each particle comprising: (i) a microparticle that comprises a pharmaceutically acceptable excipient, and (ii) nanoparticles of an antiviral, comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

Furthermore, provided herein is a method of preparing nanoparticles of an antiviral, comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

Provided herein are coated particles, each particle comprising: (i) a microparticle that comprises a pharmaceutically acceptable excipient, and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

Provided herein are nanoparticles of an antiviral, which are prepared by a method comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

Provided herein is a pharmaceutical composition, in one embodiment, a dry powder antiviral composition, comprising coated particles, each particle comprising: (i) a microparticle that comprises a pharmaceutically acceptable excipient, and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

Provided herein is a pharmaceutical composition, in one embodiment, a dry powder antiviral composition, comprising nanoparticles of an antiviral, which are prepared by a method comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

Provided herein is a batch of coated particles, each particle comprising: (i) a microparticle that comprises a pharmaceutically acceptable excipient, and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

Provided herein is a batch of nanoparticles of an antiviral, which are prepared by a method comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

Provided herein is a batch of a pharmaceutical composition, in one embodiment, a dry powder antiviral composition, comprising coated particles, each particle comprising: (i) a microparticle that comprises a pharmaceutically acceptable excipient, and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

Provided herein is a batch of a pharmaceutical composition, in one embodiment, a dry powder antiviral composition, comprising nanoparticles of an antiviral and a pharmaceutical acceptable excipient, wherein the nanoparticles are prepared by a method comprising the steps of:
a. vaporizing the antiviral at a predetermined temperature under a first predetermined vacuum pressure to form a vapor; and
b. depositing the vapor on the surface of a microparticle comprising the pharmaceutically acceptable excipient at a predetermined agitation speed and a second predetermined temperature under the predetermined vacuum pressure to form the nanoparticles on the surface of the microparticle.

Provided herein is a device for administering an antiviral by inhalation, comprising coated particles, nanoparticles, or a pharmaceutical composition provided herein, and an inhaler.

Provided herein is a kit for administering an antiviral by inhalation, comprising coated particles, nanoparticles, or a pharmaceutical composition provided herein, and an inhaler.

Provided herein is a method of treating, preventing, or ameliorating one or more symptoms of a viral infection in a subject, comprising administering to the subject in need thereof a therapeutically effective amount of coated particles, nanoparticles, or a pharmaceutical composition provided herein.

Provided herein is a method of treating, preventing, or ameliorating one or more symptoms of a severe acute respiratory syndrome in a subject, comprising administering to the subject in need thereof a therapeutically effective amount of coated particles, nanoparticles, or a pharmaceutical composition provided herein.

Provided herein is a method of reducing the severity of one or more symptoms of a viral infection in a subject, comprising administering to the subject in need thereof a therapeutically effective amount of coated particles, nanoparticles, or a pharmaceutical composition provided herein.

Provided herein is a method of reducing the severity of one or more symptoms of a severe acute respiratory syndrome in a subject, comprising administering to the subject in need thereof a therapeutically effective amount of coated particles, nanoparticles, or a pharmaceutical composition provided herein.

Provided herein is a method of inhibiting replication of a virus in a host, comprising contacting the host with an effective amount of coated particles, nanoparticles, or a pharmaceutical composition provided herein.

### BRIEF DESCRIPTION OF THE DRAWINGS

FIG. 1 illustrates the formation of nanoparticles of an antiviral on the surfaces of microparticles of a hydrophilic excipient by contacting the microparticles with a vapor of the antiviral.

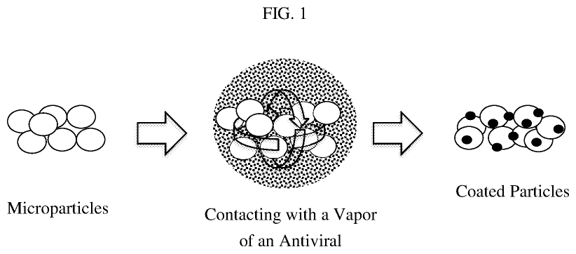

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare et al., Eds.; Academic Press: London, 2020; Handbook of Pharmaceutical Excipients, 9th ed.; Sheskey et al., Eds.; Pharmaceutical Press: London, 2020; Handbook of Pharmaceutical Additives, 3rd ed.; Ash and Ash Eds.; Synapse Information Resources: 2007; Pharmaceutical Preformulation and Formulation, 2nd ed.; Gibson Ed.; Drugs and the Pharmaceutical Sciences 199; Informa Healthcare: New York, NY, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “batch” refers to a defined quantity of a compound, material, or drug product processed in a process or series of processes so that it is homogeneous within specified limits. To complete certain stages of manufacture, it may be necessary to divide a batch into a number of sub-batches, which are later brought together to form a final homogeneous batch. In the case of continuous manufacture, the batch corresponds to a defined fraction of the production, characterized by its intended homogeneity. In manufacturing a drug product, synthetic intermediates and the drug product are each identified by a batch number.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as described herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., 1H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “13C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound described herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

When a compound described herein contains an acidic or basic moiety, it can be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011.

Suitable acids for use in the preparation of pharmaceutically acceptable salts of a compound described herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound described herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

### Coated Particles and Nanoparticles

In one embodiment, provided herein is a coated particle comprising: (i) a microparticle that comprises a pharmaceutically acceptable excipient, and (ii) nanoparticles of an antiviral; wherein the surface of the microparticle is coated with the nanoparticles.

In certain embodiments, the surface of the microparticle is coated with a layer of the nanoparticles. In certain embodiments, the surface of the microparticle is substantially coated with a layer of the nanoparticles. In certain embodiments, the surface of the microparticle is coated with a thin layer of the nanoparticles. In certain embodiments, the surface of the microparticle is substantially coated with a thin layer of the nanoparticles. In certain embodiments, the surface of the microparticle is coated with a single layer of the nanoparticles. In certain embodiments, the surface of the microparticle is substantially coated with a single layer of the nanoparticles.

In certain embodiments, the pharmaceutically acceptable excipient in the coated particle provided herein is hydrophilic. In certain embodiments, the pharmaceutically acceptable excipient in the coated particle provided herein is water-soluble. In certain embodiments, the pharmaceutically acceptable excipient in the coated particle provided herein is inhalation-grade.

In certain embodiments, the pharmaceutically acceptable excipient in the coated particle provided herein is a sugar. In certain embodiments, the pharmaceutically acceptable excipient in the coated particle provided herein is dextrose, fructose, glucose, lactose, maltose, molasses, sucrose, trehalose, or a mixture thereof. In certain embodiments, the pharmaceutically acceptable excipient in the coated particle provided herein is dextrose, glucose, lactose, sucralose, sucrose, or a mixture thereof. In certain embodiments, the pharmaceutically acceptable excipient in the coated particle provided herein is dextrose. In certain embodiments, the pharmaceutically acceptable excipient in the coated particle provided herein is fructose. In certain embodiments, the pharmaceutically acceptable excipient in the coated particle provided herein is glucose. In certain embodiments, the pharmaceutically acceptable excipient in the coated particle provided herein is lactose. In certain embodiments, the pharmaceutically acceptable excipient in the coated particle provided herein is lactose monohydrate. In certain embodiments, the pharmaceutically acceptable excipient in the coated particle provided herein is anhydrous lactose. In certain embodiments, the pharmaceutically acceptable excipient in the coated particle provided herein is inhalation-grade lactose. In certain embodiments, the pharmaceutically acceptable excipient in the coated particle provided herein is inhalation-grade lactose monohydrate. In certain embodiments, the pharmaceutically acceptable excipient in the coated particle provided herein is inhalation-grade anhydrous lactose. In certain embodiments, the pharmaceutically acceptable excipient in the coated particle provided herein is maltose. In certain embodiments, the pharmaceutically acceptable excipient in the coated particle provided herein is molasses. In certain embodiments, the pharmaceutically acceptable excipient in the coated particle provided herein is sucrose. In certain embodiments, the pharmaceutically acceptable excipient in the coated particle provided herein is trehalose.

In certain embodiments, the pharmaceutically acceptable excipient in the coated particle provided herein is a sugar alcohol. In certain embodiments, the pharmaceutically acceptable excipient in the coated particle provided herein is arabitol, erythritol, fucitol, galactitol, iditol, inositol, isomalt, lactitol, maltitol, maltotritol, mannitol, ribitol, sorbitol, threitol, volemitol, xylitol, or a mixture thereof. In certain embodiments, the pharmaceutically acceptable excipient in the coated particle provided herein is erythritol, lactitol, maltitol, mannitol, sorbitol, xylitol, or a mixture thereof. In certain embodiments, the pharmaceutically acceptable excipient in the coated particle provided herein is arabitol. In certain embodiments, the pharmaceutically acceptable excipient in the coated particle provided herein is erythritol. In certain embodiments, the pharmaceutically acceptable excipient in the coated particle provided herein is fucitol. In certain embodiments, the pharmaceutically acceptable excipient in the coated particle provided herein is galactitol. In certain embodiments, the pharmaceutically acceptable excipient in the coated particle provided herein is iditol. In certain embodiments, the pharmaceutically acceptable excipient in the coated particle provided herein is inositol. In certain embodiments, the pharmaceutically acceptable excipient in the coated particle provided herein is isomalt. In certain embodiments, the pharmaceutically acceptable excipient in the coated particle provided herein is lactitol. In certain embodiments, the pharmaceutically acceptable excipient in the coated particle provided herein is maltitol. In certain embodiments, the pharmaceutically acceptable excipient in the coated particle provided herein is maltotritol. In certain embodiments, the pharmaceutically acceptable excipient in the coated particle provided herein is mannitol. In certain embodiments, the pharmaceutically acceptable excipient in the coated particle provided herein is D-mannitol. In certain embodiments, the pharmaceutically acceptable excipient in the coated particle provided herein is inhalation-grade mannitol. In certain embodiments, the pharmaceutically acceptable excipient in the coated particle provided herein is inhalation-grade D-mannitol. In certain embodiments, the pharmaceutically acceptable excipient in the coated particle provided herein is ribitol. In certain embodiments, the pharmaceutically acceptable excipient in the coated particle provided herein is sorbitol. In certain embodiments, the pharmaceutically acceptable excipient in the coated particle provided herein is threitol. In certain embodiments, the pharmaceutically acceptable excipient in the coated particle provided herein is volemitol. In certain embodiments, the pharmaceutically acceptable excipient in the coated particle provided herein is xylitol.

In certain embodiments, the microparticle in the coated particle provided herein has various shapes, including, but not limited to, a sphere, spheroid, platelet, fibril, or fiber. In certain embodiments, the microparticle in the coated particle provided herein is substantially spherical. In certain embodiments, the microparticle in the coated particle provided herein is spherical. In certain embodiments, the microparticle in the coated particle provided herein is spheroidal.

In certain embodiments, the microparticle in the coated particle provided herein has an average particle size (D50) ranging from about 1 to about 1,000 μm, from about 2 to about 500 μm, from about 5 to about 250 μm, or from about 10 to about 250 μm. In certain embodiments, the microparticle in the coated particle provided herein has an average particle size ranging from about 1 to about 1,000 μm. In certain embodiments, the microparticle in the coated particle provided herein has an average particle size ranging from about 2 to about 500 μm. In certain embodiments, the microparticle in the coated particle provided herein has an average particle size ranging from about 5 to about 250 μm. In certain embodiments, the microparticle in the coated particle provided herein has an average particle size ranging from about 10 to about 250 μm. In certain embodiments, the microparticle in the coated particle provided herein has an average particle size of about 10, about 20, about 30, about 40, about 50, about 75, about 100, about 150, about 200, about 250, or about 300 μm.

In another embodiment, provided herein is a coated particle comprising: (i) a microparticle that comprises mannitol and (ii) nanoparticles of an antiviral; wherein the surface of the mannitol microparticle is coated with the nanoparticles.

In yet another embodiment, provided herein is a coated particle consisting of: (i) a microparticle of mannitol and (ii) nanoparticles of an antiviral; wherein the surface of the mannitol microparticle is coated with the nanoparticles.

In one embodiment, the microparticle comprises mannitol. In another embodiment, the microparticle comprises inhalation-grade mannitol.

In certain embodiments, the surface of the mannitol microparticle is coated with a layer of the nanoparticles. In certain embodiments, the surface of the mannitol microparticle is substantially coated with a layer of the nanoparticles. In certain embodiments, the surface of the mannitol microparticle is coated with a single layer of the nanoparticles. In certain embodiments, the surface of the mannitol microparticle is substantially coated with a single layer of the nanoparticles.

In certain embodiments, the mannitol microparticle in the coated particle provided herein has various shapes, including, but not limited to, a sphere, spheroid, platelet, fibril, or fiber. In certain embodiments, the mannitol microparticle in the coated particle provided herein is substantially spherical. In certain embodiments, the mannitol microparticle in the coated particle provided herein is spherical. In certain embodiments, the mannitol microparticle in the coated particle provided herein is spheroidal.

In certain embodiments, the mannitol microparticle in the coated particle provided herein has an average particle size ranging from about 1 to about 1,000 μm, from about 2 to about 500 μm, from about 5 to about 250 μm, or from about 10 to about 250 μm. In certain embodiments, the mannitol microparticle in the coated particle provided herein has an average particle size ranging from about 1 to about 1,000 μm. In certain embodiments, the mannitol microparticle in the coated particle provided herein has an average particle size ranging from about 2 to about 500 μm. In certain embodiments, the mannitol microparticle in the coated particle provided herein has an average particle size ranging from about 5 to about 250 μm. In certain embodiments, the mannitol microparticle in the coated particle provided herein has an average particle size ranging from about 10 to about 250 μm. In certain embodiments, the mannitol microparticle in the coated particle provided herein has an average particle size of about 10, about 20, about 30, about 40, about 50, about 75, about 100, about 150, about 200, about 250, or about 300 μm.

In yet another embodiment, provided herein is a coated particle comprising: (i) a microparticle that comprises lactose and (ii) nanoparticles of an antiviral; wherein the surface of the lactose microparticle is coated with the nanoparticles.

In still another embodiment, provided herein is a coated particle consisting of: (i) a microparticle of lactose and (ii) nanoparticles of an antiviral; wherein the surface of the lactose microparticle is coated with the nanoparticles.

In one embodiment, the microparticle comprises lactose monohydrate. In another embodiment, the microparticle comprises anhydrous lactose.

In one embodiment, the microparticle comprises inhalation-grade lactose. In another embodiment, the microparticle comprises inhalation-grade lactose monohydrate. In yet another embodiment, the microparticle comprises inhalation-grade anhydrous lactose.

In certain embodiments, the surface of the lactose microparticle is coated with a layer of the nanoparticles. In certain embodiments, the surface of the lactose microparticle is substantially coated with a layer of the nanoparticles. In certain embodiments, the surface of the lactose microparticle is coated with a single layer of the nanoparticles. In certain embodiments, the surface of the lactose microparticle is substantially coated with a single layer of the nanoparticles.

In certain embodiments, the lactose microparticle in the coated particle provided herein has various shapes, including, but not limited to, a sphere, spheroid, platelet, fibril, or fiber. In certain embodiments, the lactose microparticle in the coated particle provided herein is substantially spherical. In certain embodiments, the lactose microparticle in the coated particle provided herein is spherical. In certain embodiments, the lactose microparticle in the coated particle provided herein is spheroidal.

In certain embodiments, the lactose microparticle in the coated particle provided herein has an average particle size ranging from about 1 to about 1,000 μm, from about 2 to about 500 μm, from about 5 to about 250 μm, or from about 10 to about 250 μm. In certain embodiments, the lactose microparticle in the coated particle provided herein has an average particle size ranging from about 1 to about 1,000 μm. In certain embodiments, the lactose microparticle in the coated particle provided herein has an average particle size ranging from about 2 to about 500 μm. In certain embodiments, the lactose microparticle in the coated particle provided herein has an average particle size ranging from about 5 to about 250 μm. In certain embodiments, the lactose microparticle in the coated particle provided herein has an average particle size ranging from about 10 to about 250 μm. In certain embodiments, the lactose microparticle in the coated particle provided herein has an average particle size of about 10, about 20, about 30, about 40, about 50, about 75, about 100, about 150, about 200, about 250, or about 300 μm.

In certain embodiments, the antiviral in the coated particle provided herein is a class II compound according to the Biopharmaceutics Classification System (BCS), i.e., a BCS class II compound. In certain embodiments, the antiviral in the coated particle provided herein is a BCS class III compound. In certain embodiments, the antiviral in the coated particle provided herein is a BCS class IV compound.

In certain embodiments, the antiviral in the coated particle provided herein has a low solubility to the Biopharmaceutics Classification System. In certain embodiments, the antiviral in the coated particle provided herein has a low permeability to the Biopharmaceutics Classification System. In certain embodiments, the antiviral in the coated particle provided herein has a low solubility and a low permeability to the Biopharmaceutics Classification System.

In certain embodiments, the antiviral in the coated particle provided herein is a solid. In certain embodiments, the antiviral in the coated particle provided herein has a melting point ranging from about 30 to about 300° C., from about 50 to about 200° C., from about 70 to about 150° C., from about 150 to about 300° C., or from about 100 to about 150° C. In certain embodiments, the antiviral in the coated particle provided herein has a melting point ranging from about 30 to about 300° C. In certain embodiments, the antiviral in the coated particle provided herein has a melting point ranging from about 50 to about 200° C. In certain embodiments, the antiviral in the coated particle provided herein has a melting point ranging from about 70 to about 150° C. In certain embodiments, the antiviral in the coated particle provided herein has a melting point ranging from about 150 to about 300° C. In certain embodiments, the antiviral in the coated particle provided herein has a melting point ranging from about 100 to about 150° C.

In one embodiment, the antiviral in the coated particle provided herein is remdesivir, 2-ethylbutyl ((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy)phosphoryl)-L-alaninate, the structure of which is shown below; or an isotopic variant thereof; or a pharmaceutically acceptable salt, solvate, or hydrate.

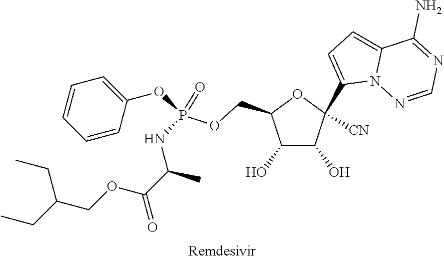 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In another embodiment, the antiviral in the coated particle provided herein is GS-441524, (2R,3R,4S,5R)-2-(4-aminopyrrolo[2,1-f][1,2,4]triazin-7-yl)-3,4-dihydroxy-5-(hydroxymethyl)tetrahydrofuran-2-carbonitrile, the structure of which is shown below; or an isotopic variant thereof; or a pharmaceutically acceptable salt, solvate, or hydrate.

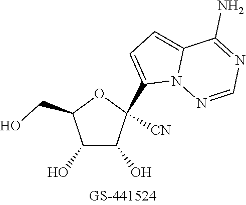 [SMILES: N#C[C@@]1(c2ccc3c(N)ncnn23)O[C@H](CO)[C@@H](O)[C@H]1O]

In one embodiment, provided herein is a coated particle comprising: (i) a microparticle that comprises a pharmaceutically acceptable excipient, and (ii) nanoparticles of remdesivir; wherein the surface of the microparticle is coated with the nanoparticles.

In another embodiment, provided herein is a coated particle comprising: (i) a microparticle that comprises mannitol, and (ii) nanoparticles of remdesivir; wherein the surface of the microparticle is coated with the nanoparticles.

In yet another embodiment, provided herein is a coated particle comprising: (i) a microparticle that comprises lactose, and (ii) nanoparticles of remdesivir; wherein the surface of the microparticle is coated with the nanoparticles.

In one embodiment, provided herein is a coated particle consisting of: (i) a microparticle that comprises a pharmaceutically acceptable excipient, and (ii) nanoparticles of remdesivir; wherein the surface of the microparticle is coated with the nanoparticles.

In another embodiment, provided herein is a coated particle consisting of: (i) a microparticle that comprises mannitol, and (ii) nanoparticles of remdesivir; wherein the surface of the microparticle is coated with the nanoparticles.

In yet another embodiment, provided herein is a coated particle consisting of: (i) a microparticle that comprises lactose, and (ii) nanoparticles of remdesivir; wherein the surface of the microparticle is coated with the nanoparticles.

In certain embodiments, the nanoparticles in the coated particle provided herein have an average particle size ranging from about 1 to about 5,000 nm, from about 10 to about 3,000 nm, from about 20 to about 3,000 nm, from about 50 to about 3,000 nm, from about 100 to about 3,000 nm, or from about and 500 to about 3,000 nm. In certain embodiments, the nanoparticles in the coated particle provided herein have an average particle size ranging from about 1 to about 5,000 nm. In certain embodiments, the nanoparticles in the coated particle provided herein have an average particle size ranging from about 10 to about 3,000 nm. In certain embodiments, the nanoparticles in the coated particle provided herein have an average particle size ranging from about 20 to about 3,000 nm. In certain embodiments, the nanoparticles in the coated particle provided herein have an average particle size ranging from about 50 to about 3,000 nm. In certain embodiments, the nanoparticles in the coated particle provided herein have an average particle size ranging from about 100 to about 3,000 nm. In certain embodiments, the nanoparticles in the coated particle provided herein have an average particle size ranging about and 500 to about 3,000 nm.

In certain embodiments, the nanoparticles in the coated particle provided herein have an average particle size ranging from about 1 to about 900 nm, from about 1 to about 500 nm, from about 1 to about 200 nm, from about 2 to about 200 nm, from about 5 to about 200 nm, from about 10 to about 200 nm, or from about 10 to about 100 nm. In certain embodiments, the nanoparticles in the coated particle provided herein have an average particle size ranging from about 1 to about 900 nm. In certain embodiments, the nanoparticles in the coated particle provided herein have an average particle size ranging from about 1 to about 500 nm. In certain embodiments, the nanoparticles in the coated particle provided herein have an average particle size ranging from about 1 to about 200 nm. In certain embodiments the nanoparticles in the coated particle provided herein have an average particle size ranging from about 1 to about 50 nm. In certain embodiments, the nanoparticles in the coated particle provided herein have an average particle size ranging from about 2 to about 200 nm. In certain embodiments, the nanoparticles in the coated particle provided herein have an average particle size ranging from about 5 to about 200 nm. In certain embodiments, the nanoparticles in the coated particle provided herein have an average particle size ranging from about 10 to about 200 nm. In certain embodiments, the nanoparticles in the coated particle provided herein have an average particle size ranging from about 10 to about 100 nm.

In certain embodiments, the nanoparticles in the coated particle provided herein are formed on the surface of the microparticle. In certain embodiments, the nanoparticles in the coated particle provided herein are formed on the surface of the microparticle by organic vapor phase deposition. See, e.g., Baldo et al., Adv. Mater. 1998, 10, 1505-14.

In certain embodiments, the percentage of the nanoparticles in the coated particle is ranging from about 0.1 to about 50% by weight, from about 0.1 to about 40% by weight, from about 0.1 to about 30% by weight, from about 0.1 to about 25% by weight, about 0.2 to about 20% by weight, about 0.5 to about 10% by weight, or about 1 to about 10% by weight.

In certain embodiments, the percentage of the nanoparticles in the coated particle is ranging from about 0.1 to about 50% by weight. In certain embodiments, the percentage of the nanoparticles in the coated particle is ranging from about 0.1 to about 40% by weight. In certain embodiments, the percentage of the nanoparticles in the coated particle is ranging from about 0.1 to about 30% by weight. In certain embodiments, the percentage of the nanoparticles in the coated particle is ranging from about 0.1 to about 25% by weight. In certain embodiments, the percentage of the nanoparticles in the coated particle is ranging from about 0.2 to about 20% by weight. In certain embodiments, the percentage of the nanoparticles in the coated particle is ranging from about 0.5 to about 10% by weight. In certain embodiments, the percentage of the nanoparticles in the coated particle is ranging from about 1 to about 10% by weight. In certain embodiments, the percentage of the nanoparticles in the coated particle is about 1, about 2, about 3, about 4, about 5, about 6, about 7, about 8, about 9, about 10, about 15, or about 20% by weight.

In certain embodiments, the coated particle provided herein has an average particle size ranging from about 1 to about 1,000 μm, from about 2 to about 500 μm, from about 5 to about 250 μm, or from about 10 to about 250 μm. In certain embodiments, the coated particle provided herein has an average particle size ranging from about 1 to about 1,000 μm. In certain embodiments, the coated particle provided herein has an average particle size ranging from about 2 to about 500 μm. In certain embodiments, the coated particle provided herein has an average particle size ranging from about 5 to about 250 μm. In certain embodiments, the coated particle provided herein has an average particle size ranging from about 10 to about 250 μm. In certain embodiments, the coated particle provided herein has an average particle size of about 10, about 20, about 30, about 40, about 50, about 75, about 100, about 150, about 200, about 250, or about 300 μm.

In certain embodiments, the coated particle provided herein has an average particle size ranging from about 0.5 to about 20 μm, from about 1 to about 10 μm, or from about 1 to about 5 μm. In certain embodiments, the coated particle provided herein has an average particle size ranging from about 0.5 to about 20 μm. In certain embodiments, the coated particle provided herein has an average particle size ranging from about 1 to about 10 μm. In certain embodiments, the coated particle provided herein has an average particle size ranging from about 1 to about 5 μm. In certain embodiments, the coated particle provided herein has an average particle size of about 1, about 2, about 3, about 4, or about 5 μm.

In certain embodiments, the coated particle provided herein has a mass median aerodynamic diameter (MMAD) ranging from about 0.5 to about 20 μm, from about 1 to about 10 μm, or from about 1 to about 5 μm. In certain embodiments, the coated particle provided herein has an MMAD ranging from about 0.5 to about 20 μm. In certain embodiments, the coated particle provided herein has an MMAD ranging from about 1 to about 10 μm. In certain embodiments, the coated particle provided herein has an MMAD ranging from about 1 to about 5 μm. In certain embodiments, the coated particle provided herein has an MMAD of about 1, about 2, about 3, about 4, or about 5 μm.

In certain embodiments, the coated particle provided herein is for administration by inhalation.

### Method of Preparation

In one embodiment, provided herein is a method of preparing coated particles, each particle comprising: (i) a microparticle that comprises a pharmaceutically acceptable excipient and (ii) nanoparticles of an antiviral, comprising the steps of:
a. vaporizing the antiviral at a first predetermined temperature under a predetermined vacuum pressure to form a vapor; and
b. depositing the vapor on the surface of the microparticle at a predetermined agitation speed and a second predetermined temperature under the predetermined vacuum pressure to form the nanoparticles on the surfaces of the microparticles, thus forming the coated particles.

In another embodiment, provided herein is a method of preparing nanoparticles of an antiviral, comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

In certain embodiments, the first predetermined temperature is ranging from about to about 500° C., 50 to about 300° C., from about 50 to about 200° C., from about 100 to about 400° C., from about from about 100 to about 300° C., from about 100 to about 250° C., from about 150 to about 300° C., or from about 200 to about 300° C. In certain embodiments, the first predetermined temperature is ranging from about 50 to about 500° C. In certain embodiments, the first predetermined temperature is ranging from about 50 to about 300° C. In certain embodiments, the first predetermined temperature is ranging from about 50 to about 200° C. In certain embodiments, the first predetermined temperature is ranging from about 100 to about 400° C. In certain embodiments, the first predetermined temperature is ranging from about 100 to about 300° C. In certain embodiments, the first predetermined temperature is ranging from about 100 to about 250° C. In certain embodiments, the first predetermined temperature is ranging from about 150 to about 300° C. In certain embodiments, the first predetermined temperature is ranging from about 200 to about 300° C. In certain embodiments, the first predetermined temperature is about 50, about 75, about 100, about 125, about 150, about 175, about 200, about 250, about 250, about 275, or about 300° C.

In certain embodiments, the predetermined vacuum pressure is no greater than about 10⁻³ torr, no greater than about 10⁻⁴ torr, no greater than about 10⁻⁵ torr, no greater than about 10⁻⁶ torr, no greater than about 10⁻⁷ torr, no greater than about 10⁻⁸ torr, or no greater than about 10⁻⁹ torr. In certain embodiments, the predetermined vacuum pressure is no greater than about 10⁻³ torr. In certain embodiments, the first predetermined vacuum pressure is no greater than about 10⁻⁴ torr. In certain embodiments, the predetermined vacuum pressure is no greater than about 10⁻⁵ torr. In certain embodiments, the predetermined vacuum pressure is no greater than about 10⁻⁶ torr. In certain embodiments, the first predetermined vacuum pressure is no greater than about 10⁻⁷ torr. In certain embodiments, the predetermined vacuum pressure is no greater than about 10⁻⁸ torr. In certain embodiments, the predetermined vacuum pressure is no greater than about 10⁻⁹ torr. In certain embodiments, the predetermined vacuum pressure is about 10⁻³, about 10⁻⁴, about 10⁻⁵, about 10⁻⁶, about 10⁻⁷, about 10⁻⁸, or about 10⁻⁹ torr.

In certain embodiments, the predetermined vacuum pressure is ranging from about 10⁻³ to about 10⁻⁹ torr. In certain embodiments, the predetermined vacuum pressure is ranging from about 10⁻⁴ to about 10⁻⁸ torr. In certain embodiments, the predetermined vacuum pressure is ranging from about 10⁻⁴ to about 10⁻⁷ torr. In certain embodiments, the predetermined vacuum pressure is ranging from about 10⁻⁴ to about 10⁻⁶ torr.

In certain embodiments, the predetermined agitation speed is ranging from about to about 200 revolutions per minute (rpm), from about 20 to about 150 rpm, from about 20 to about 120 rpm, from about 20 to about 100 rpm, from about 50 to about 100 rpm, or from about to about 100 rpm. In certain embodiments, the predetermined agitation speed is ranging from about 10 to about 200 rpm. In certain embodiments, the predetermined agitation speed is ranging from about 20 to about 150 rpm. In certain embodiments, the predetermined agitation speed is ranging from about 20 to about 120 rpm. In certain embodiments, the predetermined agitation speed is ranging from about 20 to about 100 rpm. In certain embodiments, the predetermined agitation speed is ranging from about 50 to about 100 rpm. In certain embodiments, the predetermined agitation speed is ranging from about 80 to about 100 rpm. In certain embodiments, the predetermined agitation speed is about 80, about 85, about 90, about or about 100 rpm.

To efficiently deposit the vapor of an antiviral onto the surface of a microparticle of a pharmaceutically acceptable excipient, the second predetermined temperature is set to be lower than the first predetermined temperature. Thus, in certain embodiments, the second predetermined temperature is no less than about 10, about no less than about 20, no less than about 50, or no less than about 100° C. lower than the first predetermined temperature.

In certain embodiments, the second predetermined temperature is no greater than about 150° C., no greater than about 120° C., no greater than about 100° C., no greater than about ° C., no greater than about 40° C., no greater than about 35° C., no greater than about 30° C., or no greater than about 25° C. In certain embodiments, the second predetermined temperature is no greater than about 150° C. In certain embodiments, the second predetermined temperature is no greater than about 120° C. In certain embodiments, the second predetermined temperature is no greater than about 100° C. In certain embodiments, the second predetermined temperature is no greater than about 50° C. In certain embodiments, the second predetermined temperature is no greater than about 40° C. In certain embodiments, the second predetermined temperature is no greater than about 35° C. In certain embodiments, the second predetermined temperature is no greater than about 30° C. In certain embodiments, the second predetermined temperature is no greater than about 25° C.

In certain embodiments, the second predetermined temperature is ranging from about 10 to about 100° C., from about 20 to about 100° C., from about 20 to about 60° C., from about 15 to about 50° C., or from about 20 to about 40° C. In certain embodiments, the second predetermined temperature is ranging from about 10 to about 100° C. In certain embodiments, the second predetermined temperature is ranging from about 20 to about 100° C. In certain embodiments, the second predetermined temperature is ranging from about 20 to about 60° C. In certain embodiments, the second predetermined temperature is ranging from about 15 to about 50° C. In certain embodiments, the second predetermined temperature is ranging from about 20 to about 40° C. In certain embodiments, the second predetermined temperature is about 20, about 25, about 30, about 35, or about 40° C. In certain embodiments, the second predetermined temperature is ambient temperature.

Thus, in one embodiment, provided herein are coated particles, each particle comprising: (i) a microparticle that comprises a pharmaceutically acceptable excipient and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surface of the microparticle at the predetermined agitation speed and the second predetermined temperature under the predetermined vacuum pressure to form the nanoparticles on the surfaces of the microparticles, thus forming the coated particles.

In another embodiment, provided herein are nanoparticles of an antiviral, which are prepared by a method comprising the steps of:
a. vaporizing the antiviral at the first predetermined temperature under the predetermined vacuum pressure to form a vapor; and
b. depositing the vapor on the surface of a microparticle comprising a pharmaceutically acceptable excipient at the predetermined agitation speed and the second predetermined temperature under the predetermined vacuum pressure to form the nanoparticles on the surface of the microparticle.

In yet another embodiment, provided herein is a batch of coated particles, each particle comprising: (i) a microparticle that comprises a pharmaceutically acceptable excipient and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surface of the microparticle at the predetermined agitation speed and the second predetermined temperature under the predetermined vacuum pressure to form the nanoparticles on the surfaces of the microparticles, thus forming the coated particles.

In still another embodiment, provided herein is a batch of nanoparticles of an antiviral, which are prepared by a method comprising the steps of:
a. vaporizing the antiviral at the first predetermined temperature under the predetermined vacuum pressure to form a vapor; and
b. depositing the vapor on the surface of a microparticle comprising a pharmaceutically acceptable excipient at the predetermined agitation speed and the second predetermined temperature under the predetermined vacuum pressure to form the nanoparticles on the surface of the microparticle.

In one embodiment, provided herein are coated particles, each particle comprising: (i) a mannitol microparticle and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surfaces of the mannitol microparticles at the predetermined agitation speed and the second predetermined temperature under the predetermined vacuum pressure to form the nanoparticles on the surfaces of the mannitol microparticles, thus forming the coated particles.

In another embodiment, provided herein are nanoparticles of an antiviral, which are prepared by a method comprising the steps of:
a. vaporizing the antiviral at the first predetermined temperature under the predetermined vacuum pressure to form a vapor; and
b. depositing the vapor on the surface of a mannitol microparticle at the predetermined agitation speed and the second predetermined temperature under the predetermined vacuum pressure to form the nanoparticles on the surface of the mannitol microparticle.

In yet another embodiment, provided herein is a batch of coated particles, each particle comprising: (i) a mannitol microparticle and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surfaces of the mannitol microparticles at the predetermined agitation speed and the second predetermined temperature under the predetermined vacuum pressure to form the nanoparticles on the surfaces of the mannitol microparticles, thus forming the coated particles.

In still another embodiment, provided herein is a batch of nanoparticles of an antiviral, which are prepared by a method comprising the steps of:
a. vaporizing the antiviral at the first predetermined temperature under the predetermined vacuum pressure to form a vapor; and
b. depositing the vapor on the surface of a mannitol microparticle at the predetermined agitation speed and the second predetermined temperature under the predetermined vacuum pressure to form the nanoparticles on the surface of the mannitol microparticle.

In one embodiment, provided herein are coated particles, each particle comprising: (i) a lactose microparticle and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surfaces of the lactose microparticles at the predetermined agitation speed and the second predetermined temperature under the predetermined vacuum pressure to form the nanoparticles on the surfaces of the lactose microparticles, thus forming the coated particles.

In another embodiment, provided herein are nanoparticles of an antiviral, which are prepared by a method comprising the steps of:
a. vaporizing the antiviral at the first predetermined temperature under the predetermined vacuum pressure to form a vapor; and
b. depositing the vapor on the surface of a lactose microparticle at the predetermined agitation speed and the second predetermined temperature under the predetermined vacuum pressure to form the nanoparticles on the surface of the lactose microparticle.

In yet another embodiment, provided herein is a batch of coated particles, each particle comprising: (i) a lactose microparticle and (ii) nanoparticles of an antiviral; wherein the coated particles are prepared by a method comprising the steps of:
a. vaporizing the antiviral at the first predetermined temperature under the predetermined vacuum pressure to form a vapor; and
b. depositing the vapor on the surfaces of the lactose microparticles at the predetermined agitation speed and the second predetermined temperature under the predetermined vacuum pressure to form the nanoparticles on the surfaces of the lactose microparticles, thus forming the coated particles.

In still another embodiment, provided herein is a batch of nanoparticles of an antiviral, which are prepared by a method comprising the steps of:
a. vaporizing the antiviral at the first predetermined temperature under the predetermined vacuum pressure to form a vapor; and
b. depositing the vapor on the surface of a lactose microparticle at the predetermined agitation speed and the second predetermined temperature under the predetermined vacuum pressure to form the nanoparticles on the surface of the lactose microparticle.

### Pharmaceutical Compositions

In one embodiment, provided herein is a pharmaceutical composition comprising coated particles, each particle comprising: (i) a microparticle that comprises a pharmaceutically acceptable excipient and (ii) nanoparticles of an antiviral, wherein the surface of the microparticle is coated with the nanoparticles.

In another embodiment, provided herein is a pharmaceutical composition comprising coated particles, each particle comprising: (i) a microparticle that comprises a pharmaceutically acceptable excipient and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

In yet another embodiment, provided herein is a pharmaceutical composition comprising nanoparticles of an antiviral, which are prepared by a method comprising the steps of:
a. vaporizing the antiviral at a first predetermined temperature under a predetermined vacuum pressure to form a vapor; and
b. depositing the vapor on the surface of a microparticle comprising a pharmaceutically acceptable excipient at a predetermined agitation speed and a second predetermined temperature under the predetermined vacuum pressure to form the nanoparticles on the surface of the microparticle.

In yet another embodiment, provided herein is a batch of a pharmaceutical composition comprising coated particles, each particle comprising: (i) a microparticle that comprises a pharmaceutically acceptable excipient and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the microparticles at a predetermined agitation speed and a second predetermined temperature under the predetermined vacuum pressure to form the nanoparticles on the surfaces of the microparticles, thus forming the coated particles.

In still another embodiment, provided herein is a batch of a pharmaceutical composition comprising a pharmaceutical acceptable excipient and nanoparticles of an antiviral, wherein the nanoparticles are prepared by a method comprising the steps of:
a. vaporizing the antiviral at a predetermined temperature under a first predetermined vacuum pressure to form a vapor; and
b. depositing the vapor on the surface of a microparticle comprising the pharmaceutically acceptable excipient at a predetermined agitation speed and a second predetermined temperature under the predetermined vacuum pressure to form the nanoparticles on the surface of the microparticle.

In one embodiment, provided herein is a pharmaceutical composition comprising coated particles, each particle comprising: (i) a mannitol microparticle and (ii) nanoparticles of an antiviral, wherein the surface of the mannitol microparticle is coated with the nanoparticles.

In another embodiment, provided herein is a pharmaceutical composition comprising coated particles, each particle comprising: (i) a mannitol microparticle and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the mannitol microparticles at a predetermined agitation speed and a second predetermined temperature under the predetermined vacuum pressure to form the nanoparticles on the surfaces of the mannitol microparticles, thus forming the coated particles.

In yet another embodiment, provided herein is a pharmaceutical composition comprising nanoparticles of an antiviral, which are prepared by a method comprising the steps of:
a. vaporizing the antiviral at a first predetermined temperature under a predetermined vacuum pressure to form a vapor; and
b. depositing the vapor on the surface of a mannitol microparticle comprising a pharmaceutically acceptable excipient at a predetermined agitation speed and a second predetermined temperature under the predetermined vacuum pressure to form the nanoparticles on the surface of the mannitol microparticle.

In yet another embodiment, provided herein is a batch of a pharmaceutical composition comprising coated particles, each particle comprising: (i) a mannitol microparticle and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the mannitol microparticles at a predetermined agitation speed and a second predetermined temperature under the predetermined vacuum pressure to form the nanoparticles on the surfaces of the mannitol microparticles, thus forming the coated particles.

In still another embodiment, provided herein is a batch of a pharmaceutical composition comprising mannitol and nanoparticles of an antiviral, wherein the nanoparticles are prepared by a method comprising the steps of:
a. vaporizing the antiviral at a predetermined temperature under a first predetermined vacuum pressure to form a vapor; and
b. depositing the vapor on the surface of a mannitol microparticle at a predetermined agitation speed and a second predetermined temperature under the predetermined vacuum pressure to form the nanoparticles on the surface of the mannitol microparticle.

In one embodiment, provided herein is a pharmaceutical composition comprising coated particles, each particle comprising: (i) a lactose microparticle and (ii) nanoparticles of an antiviral, wherein the surface of the lactose microparticle is coated with the nanoparticles.

In another embodiment, provided herein is a pharmaceutical composition comprising coated particles, each particle comprising: (i) a lactose microparticle and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the lactose microparticles at a predetermined agitation speed and a second predetermined temperature under the predetermined vacuum pressure to form the nanoparticles on the surfaces of the lactose microparticles, thus forming the coated particles.

In yet another embodiment, provided herein is a pharmaceutical composition comprising nanoparticles of an antiviral, which are prepared by a method comprising the steps of:
a. vaporizing the antiviral at a first predetermined temperature under a predetermined vacuum pressure to form a vapor; and
b. depositing the vapor on the surface of a lactose microparticle comprising a pharmaceutically acceptable excipient at a predetermined agitation speed and a second predetermined temperature under the predetermined vacuum pressure to form the nanoparticles on the surface of the lactose microparticle.

In yet another embodiment, provided herein is a batch of a pharmaceutical composition comprising coated particles, each particle comprising: (i) a lactose microparticle and (ii) nanoparticles of an antiviral; wherein the coated particles are prepared by a method comprising the steps of:
a. vaporizing the antiviral at a first predetermined temperature under a predetermined vacuum pressure to form a vapor; and
b. depositing the vapor on the surfaces of the lactose microparticles at a predetermined agitation speed and a second predetermined temperature under the predetermined vacuum pressure to form the nanoparticles on the surfaces of the lactose microparticles, thus forming the coated particles.

In still another embodiment, provided herein is a batch of a pharmaceutical composition comprising lactose and nanoparticles of an antiviral, wherein the nanoparticles are prepared by a method comprising the steps of:
a. vaporizing the antiviral at a predetermined temperature under a first predetermined vacuum pressure to form a vapor; and
b. depositing the vapor on the surface of a lactose microparticle at a predetermined agitation speed and a second predetermined temperature under the predetermined vacuum pressure to form the nanoparticles on the surface of the lactose microparticle.

In certain embodiments, a pharmaceutical composition provided herein is formulated as a dry powder. In certain embodiments, a pharmaceutical composition provided herein is formulated as a dry powder for administration by inhalation.

The pharmaceutical compositions provided herein can each independently be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) sufficient to produce the desired therapeutic effect, in association with the required pharmaceutical excipient(s). Examples of a unit-dosage form include, but are not limited to, a capsule or blister. A multiple-dosage form is a plurality of identical unit-dosage forms packaged in a single container to be administered in a segregated unit-dosage form. Examples of a multiple-dosage form include, are not limited to, a blister pack, blister disk, blister strip, cartridge, or reservoir.

In one embodiment, a pharmaceutical composition provided herein is formulated as a capsule for administration by inhalation. In another embodiment, a pharmaceutical composition provided herein is formulated as a blister for administration by inhalation.

In one embodiment, a pharmaceutical composition provided herein is formulated as a multi-dose blister pack for administration by inhalation. In another embodiment, a pharmaceutical composition provided herein is formulated as a multi-dose blister disk for administration by inhalation. In yet another embodiment, a pharmaceutical composition provided herein is formulated as a multi-dose blister strip for administration by inhalation. In yet another embodiment, a pharmaceutical composition provided herein is formulated as a multi-dose cartridge for administration by inhalation. In still another embodiment, a pharmaceutical composition provided herein is formulated as a multi-dose reservoir for administration by inhalation.

In one embodiment, a pharmaceutical composition provided herein is formulated for administration by a dry powder inhaler. In one embodiment, the dry powder inhaler is a passive dry powder inhaler. In another embodiment, the dry powder inhaler is an active dry powder inhaler.

The pharmaceutical compositions provided herein can each independently be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

In one embodiment, provided herein is a device for administration of an antiviral by inhalation, comprising coated particles provided herein and a dry powder inhaler.

In another embodiment, provided herein is a device for administration of an antiviral by inhalation, comprising nanoparticles provided herein and a dry powder inhaler.

In yet another embodiment, provided herein is a device for administration of an antiviral by inhalation, comprising a pharmaceutical composition provided herein and a dry powder inhaler.

In one embodiment, provided herein is a kit for administration of an antiviral by inhalation, comprising coated particles provided herein and a dry powder inhaler.

In another embodiment, provided herein is a kit for administration of an antiviral by inhalation, comprising nanoparticles provided herein and a dry powder inhaler.

In yet another embodiment, provided herein is a kit for administration of an antiviral by inhalation, comprising a pharmaceutical composition provided herein and a dry powder inhaler.

In certain embodiments, the kit further comprises instructions for administration of the antiviral.

### Method of Use

In one embodiment, provided herein is a method of treating, preventing, or ameliorating one or more symptoms of a viral infection in a subject, comprising administering to the subject in need thereof a therapeutically effective amount of a pharmaceutical composition provided herein.

In another embodiment, provided herein is a method of reducing the severity of one or more symptoms of a viral infection in a subject, comprising administering to the subject in need thereof a therapeutically effective amount of a pharmaceutical composition provided herein.

In certain embodiments, the symptom of the viral infection is fever or chill, cough, shortness of breath or difficulty breathing, fatigue, muscle or body ache, headache, loss of taste or smell, sore throat, congestion or runny nose, nausea or vomiting, or diarrhea.

In certain embodiments, the viral infection is a Coronaviridae virus infection. In certain embodiments, the viral infection is a Coronavirinae virus infection. In certain embodiments, the viral infection is a Betacoronavirus infection. In certain embodiments, the viral infection is a SARS-CoV-1, SARS-CoV-2, or MERS-CoV infection. In certain embodiments, the viral infection is a SARS-CoV-1 infection. In certain embodiments, the viral infection is a SARS-CoV-2 infection. In certain embodiments, the viral infection is a MERS-CoV infection.

In one embodiment, provided herein is a method of treating, preventing, or ameliorating one or more symptoms of a severe acute respiratory syndrome in a subject, comprising administering to the subject in need thereof a therapeutically effective amount of a pharmaceutical composition provided herein.

In another embodiment, provided herein is a method of reducing the severity of one or more symptoms of a severe acute respiratory syndrome in a subject, comprising administering to the subject in need thereof a therapeutically effective amount of a pharmaceutical composition provided herein.

In certain embodiments, the symptom of the severe acute respiratory syndrome is fever or chill, cough, shortness of breath or difficulty breathing, fatigue, muscle or body ache, headache, loss of taste or smell, sore throat, congestion or runny nose, nausea or vomiting, or diarrhea.

In certain embodiments, the severe acute respiratory syndrome is caused by a Coronaviridae virus. In certain embodiments, the severe acute respiratory syndrome is caused by a Coronavirinae virus. In certain embodiments, the severe acute respiratory syndrome is caused by a Betacoronavirus. In certain embodiments, the severe acute respiratory syndrome is caused by a SARS-CoV-1, SARS-CoV-2, or MERS-CoV. In certain embodiments, the severe acute respiratory syndrome is caused by a SARS-CoV-1. In certain embodiments, the severe acute respiratory syndrome is caused by a SARS-CoV-2. In certain embodiments, the severe acute respiratory syndrome is caused by a MERS-CoV.

In certain embodiments, a pharmaceutical composition provided herein is administered by inhalation. In certain embodiments, a pharmaceutical composition provided herein is administered by inhalation with a dry powder inhaler.

In certain embodiments, the subject is a mammal. In certain embodiments, the subject is a human.

In certain embodiments, the therapeutically effective amount is ranging from about 0.5 to about 10 mg/kg/day or from about 1 to about 5 mg/kg/day. In one embodiment, the therapeutically effective amount is ranging from about 0.5 to about 10 mg/kg/day. In another embodiment, the therapeutically effective amount is ranging from about 1 to about 5 mg/kg/day. In yet another embodiment, the therapeutically effective amount is about 1, about 2, about 3, about 4, or about 5 mg/kg/day.

In certain embodiments, the therapeutically effective amount is ranging from about 10 to about 500 mg per day, from about 20 to about 250 mg per day, or from about 50 to about 200 mg per day. In one embodiment, the therapeutically effective amount is ranging from about 10 to about 500 mg per day. In another embodiment, the therapeutically effective amount is ranging from about 20 to about 250 mg per day. In yet another embodiment, the therapeutically effective amount is ranging from about 50 to about 200 mg per day. In still another embodiment, the therapeutically effective amount is about 50, about 75, about 100, about 125, about 150, about 175, about 200, or about 250 mg per day.

A pharmaceutical composition provided herein can be administered once daily (QD) or divided into multiple daily doses such as twice daily (BID), and three times daily (TID). A pharmaceutical composition provided herein can be administered repetitively if necessary, for example, until the subject experiences stable disease or regression, or until the patient experiences disease progression or unacceptable toxicity.

A pharmaceutical composition provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a pharmaceutical composition provided herein is independent of the route of administration of a second therapy. In one embodiment, a pharmaceutical composition provided herein is administered by inhalation, and the second therapy is administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form.

In one embodiment, provided herein is a method of inhibiting replication of a virus in a host, comprising contacting the host with an effective amount of a pharmaceutical composition provided herein.

In certain embodiments, the virus is a Coronaviridae virus. In certain embodiments, the virus is a Coronavirinae virus. In certain embodiments, the virus is a Betacoronavirus. In certain embodiments, the virus is a SARS-CoV-1, SARS-CoV-2, or MERS-CoV. In certain embodiments, the virus is a SARS-CoV-1. In certain embodiments, the virus is a SARS-CoV-2. In certain embodiments, the virus is a MERS-CoV.

In certain embodiments, the host is a virus-infected human. In certain embodiments, the host is a virus-infected human cell.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); nm (nanometer); h (hour or hours); min (minutes); rpm (revolutions per minute); and HPLC (high performance liquid chromatography).

Unless otherwise indicated, all temperatures are expressed in ° C. (degrees Centigrade). All procedures are conducted at room temperature unless otherwise specified. Methodologies illustrated herein are intended to exemplify the applicable technologies through the use of specific examples and are not indicative of the scope of the disclosure.

### Example 1

### Preparation of Mannitol Microparticles Surface-Coated with Ibuprofen Nanoparticles

Mannitol microparticles surface-coated with ibuprofen nanoparticles were prepared using physical vapor deposition (PVD) technology. See, e.g., Baldo et al., Adv. Mater. 1998, 10, 1505-14; KR 100644219 B1; and WO 2021/168043 A1, the disclosure of each of which is incorporated herein by reference in its entirety. Briefly, ibuprofen (10 g) (TCI) was loaded in a sample feed container of a nanosizer. Mannitol (6,000 g) was loaded onto a coating pan. The nanosizer was conditioned to a vacuum pressure of no greater than 0.01 torr using a vacuum pump. The sample feed chamber inside the nanosizer was heated sequentially to 100° C. for 1 h, 150° C. for 1 h, and then 200° C. for 2 h under vacuum to vaporize ibuprofen while the coating pan was agitated at 90 rpm at ambient temperature without active heating. Ibuprofen vapor coated agitated mannitol microparticles on surface upon contact to form ibuprofen nanoparticles. Upon completion, the nanosizer was pressurized to the atmospheric pressure and the coating pan was removed to collect mannitol microparticles surface-coated with ibuprofen nanoparticles. The mannitol microparticles surface-coated with ibuprofen nanoparticles thus prepared are calculated to have a drug-loading of 2% by weight.

### Example 2

### Preparation of Mannitol Microparticles Surface-Coated with Ibuprofen Nanoparticles

Mannitol microparticles surface-coated with ibuprofen nanoparticles were also prepared using physical vapor deposition (PVD) technology. See, e.g., Baldo et al., Adv. Mater. 1998, 10, 1505-14; KR 100644219 B1; and WO 2021/168043 A1, the disclosure of each of which is incorporated herein by reference in its entirety. Briefly, ibuprofen (30 g) (TCI) was loaded in a sample feed container of a nanosizer. Mannitol (1,000 g) was loaded onto a coating pan. The nanosizer was conditioned to a vacuum pressure of no greater than 0.01 torr using a vacuum pump. The feed chamber inside the nanosizer was heated sequentially to 100° C. for 1 h, 150° C. for 1 h, and then 200° C. for 1 h under vacuum to vaporize ibuprofen while the coating pan was agitated at 90 rpm at ambient temperature without active heating. Ibuprofen vapor coated agitated mannitol microparticles on surface upon contact to form ibuprofen nanoparticles. Upon completion, the nanosizer was pressurized to the atmospheric pressure and the coating pan was removed to collect mannitol microparticles surface-coated with ibuprofen nanoparticles. The mannitol microparticles surface-coated with ibuprofen nanoparticles thus prepared are calculated to have a drug-loading of 9% by weight.

### Example 3

### Determination of Particle Size Distribution of Ibuprofen Nanoparticles by Light Diffraction

Mannitol microparticles surface-coated with ibuprofen nanoparticles (1 g) are dissolved in water (1 mL). The resulting homogenized aqueous solution containing ibuprofen nanoparticles is analyzed for particle size distribution using a Malvern MASTERSIZER 3000 particle size analyzer. Three different samples are analyzed separately.

### Example 4

### Particle Size Analysis of Ibuprofen Nanoparticles by SEM

Mannitol microparticles surface-coated with ibuprofen nanoparticles (1 g) are dissolved in water (1 mL). The resulting homogenized aqueous solution containing ibuprofen nanoparticles is analyzed using scanning electron microscopy (SEM). Three different samples are analyzed separately.

### Example 5

### Particle Size Analysis of Ibuprofen Nanoparticles by TEM

Mannitol microparticles surface-coated with ibuprofen nanoparticles (1 g) are dissolved in water (1 mL). The resulting homogenized aqueous solution containing ibuprofen nanoparticles is analyzed using transmission electron microscopy (TEM). Three different samples are analyzed separately.

### Example 6

### Purity Determination of Ibuprofen Using HPLC

Mannitol microparticles surface-coated with ibuprofen nanoparticles (0.1 g) are dissolved in water/acetonitrile (1 mL, 6:4). The sample is sonicated for 1-3 min. The sample is then analyzed by HPLC using a C18 column. Three different samples are analyzed separately.

### Example 7

### Preparation of Mannitol Microparticles Surface-Coated with Remdesivir Nanoparticles (5% Drug Loading)

Mannitol microparticles surface-coated with remdesivir nanoparticles were prepared using physical vapor deposition (PVD) technology. See, e.g., Baldo et al., Adv. Mater. 1998, 10, 1505-14; KR 100644219 B1; and WO 2021/168043 A1, the disclosure of each of which is incorporated herein by reference in its entirety. Remdesivir (2.5 g) was nanosized at 140° C. under 4×10⁻⁶ torr as described in WO 2021/168043 A1, the disclosure of which is incorporated herein by reference in its entirety. The vaporized remdesivir was in-situ coated on mannitol (50 g) at an agitation spend of 120 rpm at 140° C. The coated microparticles were determined by HPLC to have a drug loading of 5% with no impurities over 0.15% by area.

Three remdesivir nanoparticle samples for a particle sizer SALD-2300 (Shimadzu) were prepared by adding the mannitol microparticles surface-coated with remdesivir nanoparticles (100 mg) in 20 mL of a 0.1% TWEEN 80 solution in water. The remdesivir nanoparticle samples were then sonicated for 8 min. Triplicate measurements of each remdesivir nanoparticle sample were made. The remdesivir nanoparticles were determined to have a D50 of 750 nm. The remdesivir nanoparticles were also analyzed using a scattering electron microscope (SEM) at 20 kV with a 3.5 spot size to have an average size of 561 nm (n=15).

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A coated particle comprising: (i) a microparticle that comprises a pharmaceutically acceptable excipient and (ii) nanoparticles of an antiviral; wherein the surface of the microparticle is coated with the nanoparticles.

2. The coated particle of claim 1, wherein the surface of the microparticle is coated with a layer of the nanoparticles.

3. The coated particle of claim 1 or 2, wherein the surface of the microparticle is coated with a single layer of the nanoparticles.

4. The coated particle of any one of claims 1 to 3, wherein the coated particle is prepared by a method comprising the steps of:
a. vaporizing the antiviral at a first predetermined temperature under a predetermined vacuum pressure to form a vapor; and
b. depositing the vapor on the surface of the microparticle at a predetermined agitation speed and a second predetermined temperature under the predetermined vacuum pressure to form the nanoparticles on the surfaces of the microparticles, thus forming the coated particle.

5. The coated particle of claim 4, wherein the first predetermined temperature is ranging from about 30 to about 300° C.

6. The coated particle of claim 4 or 5, wherein the predetermined vacuum pressure is no greater than about 10⁻³ torr.

7. The coated particle of any one of claims 4 to 6, wherein the predetermined agitation speed is ranging from about 10 to about 200 rpm.

8. The coated particle of any one of claims 4 to 7, wherein the second predetermined temperature is no greater than about 120° C.

9. The coated particle of any one of claims 4 to 8, wherein the second predetermined temperature is ranging from about 20 to about 100° C.

10. The coated particle of any one of claims 1 to 9, wherein the pharmaceutically acceptable excipient is a hydrophilic excipient.

11. The coated particle of any one of claims 1 to 10, wherein the pharmaceutically acceptable excipient is a sugar alcohol.

12. The coated particle of any one of claims 1 to 11, wherein the pharmaceutically acceptable excipient is arabitol, erythritol, fucitol, galactitol, iditol, inositol, isomalt, lactitol, maltitol, maltotritol, mannitol, ribitol, sorbitol, threitol, volemitol, xylitol, or a mixture thereof.

13. The coated particle of any one of claims 1 to 12, wherein the pharmaceutically acceptable excipient is mannitol, sorbitol, xylitol, or a mixture thereof.

14. The coated particle of any one of claims 1 to 13, wherein the pharmaceutically acceptable excipient is mannitol.

15. The coated particle of any one of claims 1 to 14, wherein the pharmaceutically acceptable excipient is inhalation-grade mannitol.

16. The coated particle of any one of claims 1 to 10, wherein the pharmaceutically acceptable excipient is a sugar.

17. The coated particle of any one of claims 1 to 10 and 16, wherein the pharmaceutically acceptable excipient is dextrose, fructose, glucose, lactose, maltose, molasses, sucrose, trehalose, or a mixture thereof.

18. The coated particle of any one of claims 1 to 10, 16, and 17, wherein the pharmaceutically acceptable excipient is lactose.

19. The coated particle of any one of claims 1 to 10 and 16 to 18, wherein the pharmaceutically acceptable excipient is inhalation-grade lactose monohydrate.

20. The coated particle of any one of claims 1 to 10 and 16 to 18, wherein the pharmaceutically acceptable excipient is inhalation-grade anhydrous lactose.

21. The coated particle of any one of claims 1 to 20, wherein the microparticle has an average particle size ranging from about 1 to about 500 μm.

22. The coated particle of any one of claims 1 to 21, wherein the antiviral is a BCS class II compound.

23. The coated particle of any one of claims 1 to 21, wherein the antiviral is a BCS class IV compound.

24. The coated particle of any one of claims 1 to 23, wherein the antiviral is 2-ethylbutyl ((S)-(((2R,3S,4R,5R)-5-(4-aminopyrrolo[2,1-f][1,2,4]triazin-7-yl)-5-cyano-3,4-dihydroxytetrahydrofuran-2-yl)methoxy)(phenoxy)phosphoryl)-L-alaninate, or an isotopic variant thereof; or a pharmaceutically acceptable salt, solvate, or hydrate.

25. The coated particle of any one of claims 1 to 23, wherein the antiviral is GS-441524, (2R,3R,4S,5R)-2-(4-aminopyrrolo[2,1-f][1,2,4]triazin-7-yl)-3,4-dihydroxy-5-(hydroxymethyl)tetrahydrofuran-2-carbonitrile, or an isotopic variant thereof; or a pharmaceutically acceptable salt, solvate, or hydrate.

26. The coated particle of any one of claims 1 to 25, wherein the nanoparticles have an average particle size ranging from about 1 to about 5,000 nm.

27. The coated particle of any one of claims 1 to 26, wherein the coated particle contains the nanoparticles in an amount ranging from about 0.1 to about 50% by weight.

28. The coated particle of any one of claims 1 to 27, wherein the coated particle has an average particle size ranging from about 1 to about 1,000 μm.

29. The coated particle of any one of claims 1 to 28, wherein the coated particle has a mass median aerodynamic diameter ranging from about 0.5 to about 20 μm.

30. The coated particle of any one of claims 1 to 29, wherein the coated particle has a mass median aerodynamic diameter ranging from about 1 to about 5 μm.

31. A pharmaceutical composition comprising a coated particle of any one of claims 1 to 30.

32. The pharmaceutical composition of claim 31, wherein the pharmaceutical composition is for administration by inhalation.

33. The pharmaceutical composition of claim 31 or 32, wherein the pharmaceutical composition is formulated as a dry powder.

34. The pharmaceutical composition of any one of claims 31 to 33, wherein the pharmaceutical composition is formulated as a single dosage form.

35. The pharmaceutical composition of claim 34, wherein the single dosage form is a capsule or blister.

36. The pharmaceutical composition of any one of claims 31 to 33, wherein the pharmaceutical composition is formulated as a multi-dosage form.

37. The pharmaceutical composition of any one of claim 36, wherein the multi-dosage form is a cartridge or reservoir.

38. A method of treating, preventing, or ameliorating one or more symptoms of a viral infection in a subject, comprising administering to the subject in need thereof a therapeutically effective amount of a coated particle of any one of claims 1 to 30; or a pharmaceutical composition of any one of claims 31 to 37.

39. A method of reducing the severity of one or more symptoms of a viral infection in a subject, comprising administering to the subject in need thereof a therapeutically effective amount of a coated particle of any one of claims 1 to 30; or a pharmaceutical composition of any one of claims 31 to 37.

40. The method of claim 39 or 41, wherein the viral infection is coronavirus infection.

41. The method of any one of claims 39 to 40, wherein the viral infection is SARS-CoV2 infection.

42. A method of treating, preventing, or ameliorating one or more symptoms of a severe acute respiratory syndrome in a subject, comprising administering to the subject in need thereof a therapeutically effective amount of a coated particle of any one of claims 1 to 30; or a pharmaceutical composition of any one of claims 31 to 37.

43. A method of reducing the severity of one or more symptoms of a severe acute respiratory syndrome in a subject, comprising administering to the subject in need thereof a therapeutically effective amount of a coated particle of any one of claims 1 to 30; or a pharmaceutical composition of any one of claims 31 to 37.

44. The method of claim 42 or 43, wherein the severe acute respiratory syndrome is COVID-19.

45. The method of any one of claims 39 to 44, wherein the therapeutically effective amount is ranging from about 0.5 to about 10 mg/kg/day.

46. The method of any one of claims 39 to 45, wherein the therapeutically effective amount is ranging from about 10 to about 500 mg per day.

47. The method of any one of claims 39 to 46, wherein the coated particle or pharmaceutical composition is administered once a day or twice a day.

48. The method of any one of claims 39 to 47, wherein the subject is a human.

49. A method for inhibiting replication of a virus in a host, which comprises contacting the host with an effective amount of a coated particle of any one of claims 1 to 30; or a pharmaceutical composition of any one of claims 31 to 37.

50. The method of claim 49, wherein the viral is SARS-CoV2.

51. The method of claim 49 or 50, wherein the host is a human.

52. The method of claim 49 or 50, wherein the host is a cell.
